(19) United States
(12) Patent Application Publication
(10) Pub. No.: US 2021/0386450 A1
(43) Pub. Date: Dec. 16, 2021

(54) ULTRASOUND TRANSDUCER DEVICE AND ULTRASOUND TREATMENT TOOL

(71) Applicant: OLYMPUS CORPORATION, Tokyo (JP)

(72) Inventor: Kenji UESUGI, Machida-shi (JP)

(73) Assignee: OLYMPUS CORPORATION, Tokyo (JP)

(21) Appl. No.: 17/446,093

(22) Filed: Aug. 26, 2021

Related U.S. Application Data
(63) Continuation of application No. PCT/JP2019/007320, filed on Feb. 26, 2019.

(51) Int. Cl. A61B 17/32; B06B 1/02

(52) U.S. Cl. CPC A61B 17/320068; A61B 2017/320082; A61B 2017/320095; B06B 1/02

(57) ABSTRACT

An ultrasound transducer device can include an ultrasound transducer, and first electrodes that abut second electrodes. Each first electrode can have an annular shape about a rotation axis of the ultrasound transducer device. The first electrodes can be annular and can each have different diameters. The device can also include first support members that includes a support base and first deformation portions, and each first deformation portion can protrude from the support base along the rotation axis. The first deformation portions can be annular and can each have different diameters.

### CROSS-REFERENCE TO RELATED APPLICATION

This application is a continuation of International Application No. PCT/JP2019/007320, filed on Feb. 26, 2019, the entire contents of which are incorporated herein by reference.

### BACKGROUND

### 1. Technical Field

The present disclosure relates to an ultrasound transducer device and an ultrasound treatment tool.

### 2. The Related Art

In the related art, there is known an ultrasound treatment tool that applies ultrasound energy to a region to be treated (hereinafter, described as a target portion) in body tissue to treat the target portion (e.g., see JP 2009-233329 A).

The ultrasound treatment tool described in JP 2009-233329 A includes a handpiece and an ultrasound transducer device (transducer unit).

The handpiece includes an end effector (vibration transmission member) configured to treat a target portion, a grip (handle unit) configured to support the end effector, a second support member (electrode holding member) that is provided at the grip, and a second electrode (electrode member) that is provided in the second support member.

The ultrasound transducer device is rotatably mounted to the grip about a center axis of the end effector, together with the end effector. The ultrasound transducer device includes an ultrasound transducer that is mechanically connected to the end effector to transmit generated ultrasound vibration to the end effector, a first support member (transducer cover) configured to support the ultrasound transducer, and a first electrode that is provided in the first support member. The first electrode has an annular shape to surround the center axis of the end effector, abuts on the second electrode, and thereby makes electrical connect with the second electrode.

### SUMMARY

In some embodiments, provided is an ultrasound transducer device removably and rotatably mounted to a casing. The ultrasound transducer device includes: an ultrasound transducer configured to generate ultrasound vibration to treat body tissue in a predetermined vibration direction; a plurality of first electrodes configured to abut on a plurality of second electrodes provided in the casing, each first electrode having an annular shape surrounding a rotation axis of rotation of the ultrasound transducer device, the plurality of first electrodes having annular shapes with different diameter dimensions; and a plurality of first support members that includes a support base and a plurality of first deformation portions, each first deformation portion protruding from the support base along the rotation axis so as to surround the rotation axis, the plurality of first deformation portions having annular shapes with different diameter dimensions, the plurality of first support members supporting the plurality of first electrodes. In each of the plurality of first deformation portions, a protrusion dimension from the support base is set to increase as the diameter dimension of the first deformation portion decreases, a space extending over an entire periphery in a circumferential direction around the rotation axis is provided between the first deformation portions adjacently positioned, and the first deformation portion is configured to elastically deform according to an external force to move the first electrode.

In some embodiments, an ultrasound treatment tool includes: an end effector configured to treat body tissue; a casing configured to support the end effector; and an ultrasound transducer device that is removably and rotatably mounted to the casing. The ultrasound transducer device includes: an ultrasound transducer configured to generate ultrasound vibration to treat the body tissue in a predetermined vibration direction; a plurality of first electrodes configured to abut on a plurality of second electrodes provided in the casing, each first electrode having an annular shape surrounding a rotation axis of rotation of the ultrasound transducer device, the plurality of first electrodes having annular shapes with different diameter dimensions; and a plurality of first support members that includes a support base and a plurality of first deformation portions, each first deformation portion protruding from the support base along the rotation axis so as to surround the rotation axis, the plurality of first deformation portions having annular shapes with different diameter dimensions, the plurality of first support members supporting the plurality of first electrodes. In each of the plurality of first deformation portions, a protrusion dimension from the support base is set to increase as the diameter dimension of the first deformation portion decreases, a space extending over an entire periphery in a circumferential direction around the rotation axis is provided between the first deformation portions adjacently positioned, and the first deformation portion is configured to elastically deforms according to an external force to move the first electrode.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an ultrasound treatment system according to an embodiment;

FIG. 2 is a diagram illustrating an end portion on a distal end side of an ultrasound treatment tool;

FIG. 3 is a diagram illustrating a configuration of an ultrasound transducer device;

FIG. 4 is a diagram illustrating a configuration of the ultrasound transducer device;

FIG. 5 is a diagram illustrating a configuration of the ultrasound transducer device;

FIG. 6 is a diagram illustrating a configuration of the ultrasound transducer device;

FIG. 7 is a diagram illustrating a configuration of the ultrasound transducer device;

FIG. 8 is a diagram illustrating a configuration of the ultrasound transducer device;

FIG. 9 is a diagram illustrating a configuration of the ultrasound transducer device;

FIG. 10 is a diagram illustrating a configuration of the ultrasound transducer device;

FIG. 11 is a diagram illustrating a configuration of the ultrasound transducer device;

FIG. 12 is a diagram illustrating a configuration of the ultrasound transducer device;

FIG. 13 is a diagram illustrating a configuration of the ultrasound transducer device;

FIG. 14 is a diagram illustrating a configuration of the ultrasound transducer device;

FIG. 15 is a diagram illustrating a configuration of a handpiece-side electrode unit;

FIG. 16 is a diagram illustrating a configuration of the handpiece-side electrode unit;

FIG. 17 is a diagram illustrating a configuration of the handpiece-side electrode unit;

FIG. 18 is a diagram illustrating a configuration of the handpiece-side electrode unit; and

FIG. 19 is a diagram illustrating a configuration of the handpiece-side electrode unit.

### DETAILED DESCRIPTION

Modes for carrying out the disclosure (hereinafter referred to as “embodiments”) will be described below with reference to the drawings. It should be understood that the disclosure is not limited to the embodiments described below. Furthermore, in the description of the drawings, the same portions are denoted by the same reference numerals and symbols.

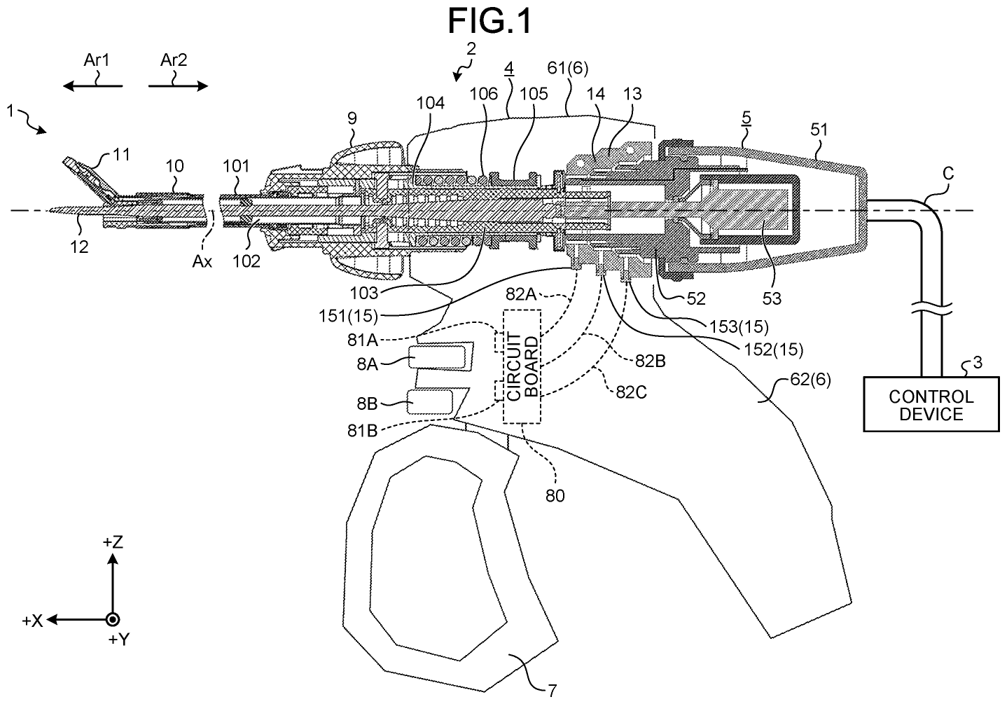

Schematic Configuration of Ultrasound Treatment System FIG. 1 is a diagram illustrating a schematic configuration of an ultrasound treatment system 1 according to the present embodiment.

The ultrasound treatment system 1 applies ultrasound energy to a region to be treated (hereinafter, described as a target portion) in body tissue to perform a treatment on the target portion. Here, examples of the treatment can include coagulation (sealing) of the target portion, incision of the target portion, and the like. As illustrated in FIG. 1, the ultrasound treatment system 1 includes an ultrasound treatment tool 2 and a control device 3.

Configuration of Ultrasound treatment tool Note that in the following description, XYZ coordinate axes of an X-axis, a Y-axis, and a Z-axis that are orthogonal to each other are used for description of the configuration of the ultrasound treatment tool 2. The X-axis is an axis parallel to a center axis Ax (FIG. 1) of a sheath 10. The center axis Ax corresponds to a rotation axis. The Y-axis is an axis orthogonal to the drawing of FIG. 1. The Z-axis is an axis extending in a vertical direction of FIG. 1. Furthermore, hereinafter, one side (+X-axis side) of the center axis Ax is referred to as a distal end side Ar1, and the other side (−X-axis side) is referred to as a proximal end side Ar2.

The ultrasound treatment tool 2 is, for example, a medical treatment tool that performs the treatment on the target portion while passing through an abdominal wall. As illustrated in FIG. 1, the ultrasound treatment tool 2 includes a handpiece 4 and an ultrasound transducer device 5.

As illustrated in FIG. 1, the handpiece 4 includes a holding case 6, a movable handle 7, first and second switches 8A and 8B, a rotation knob 9, the sheath 10, a jaw 11, a vibration transmission member 12, and a handpiece-side electrode unit 13.

The holding case 6 corresponds to a casing and supports the entire ultrasound treatment tool 2. As illustrated in FIG. 1, the holding case 6 includes a holding case body 61 that has substantially a cylindrical shape coaxial with the center axis Ax, and a fixed handle 62 that extends from the holding case body 61 to the −Z-axis side (lower side in FIG. 1) so as to be gripped by an operator such as a surgeon.

The movable handle 7 is rotatably mounted to the holding case 6. Then, the movable handle 7 receives closing operation and opening operation by the operator such as the surgeon. The closing operation or opening operation causes the movable handle 7 to rotate with respect to the holding case 6. Note that although not specifically illustrated, the movable handle 7 is engaged with a slider 105 (FIG. 1) constituting the sheath 10.

As illustrated in FIG. 1, the first and second switches 8A and 8B are provided so as to be exposed to the outside from a side surface on the distal end side Ar1 of the holding case 6.

Then, the first switch 8A receives a setting operation for a first energy output mode by the operator such as the surgeon. In addition, the second switch 8B receives a setting operation for a second energy output mode by the operator such as the surgeon. Note that the second energy output mode is an energy output mode in which a treatment different from a treatment to be performed in the first energy output mode is performed.

As illustrated in FIG. 1, in the holding case 6, a circuit board 80 on which first and second switch elements 81A and 81B are mounted is provided. Note that the first switch element 81A is a switch element configured to detect the setting operation for the first energy output mode to the first switch 8A. In addition, the second switch element 81B is a switch element configured to detect the setting operation for the second energy output mode to the second switch 8B.

As illustrated in FIG. 1, to the circuit board 80, first wiring 82A that has one end electrically connected to the first switch element 81A, second wiring 82B that has one end electrically connected to the second switch element 81B, and third wiring 82C for ground that has one end connected to a common terminal for ground are connected.

The rotation knob 9 has substantially a cylindrical shape coaxial with the center axis Ax, and is mounted to an end on the distal end side Ar1 of the holding case body 61 so as to be rotatable about the center axis Ax, as illustrated in FIG. 1. Then, the rotation knob 9 receives a rotation operation by the operator such as the surgeon. The rotation operation causes the rotation knob 9 to rotate about the center axis Ax relative to the holding case body 61. Furthermore, the rotation of the rotation knob 9 causes the jaw 11 and the vibration transmission member 12 to rotate about the center axis Ax.

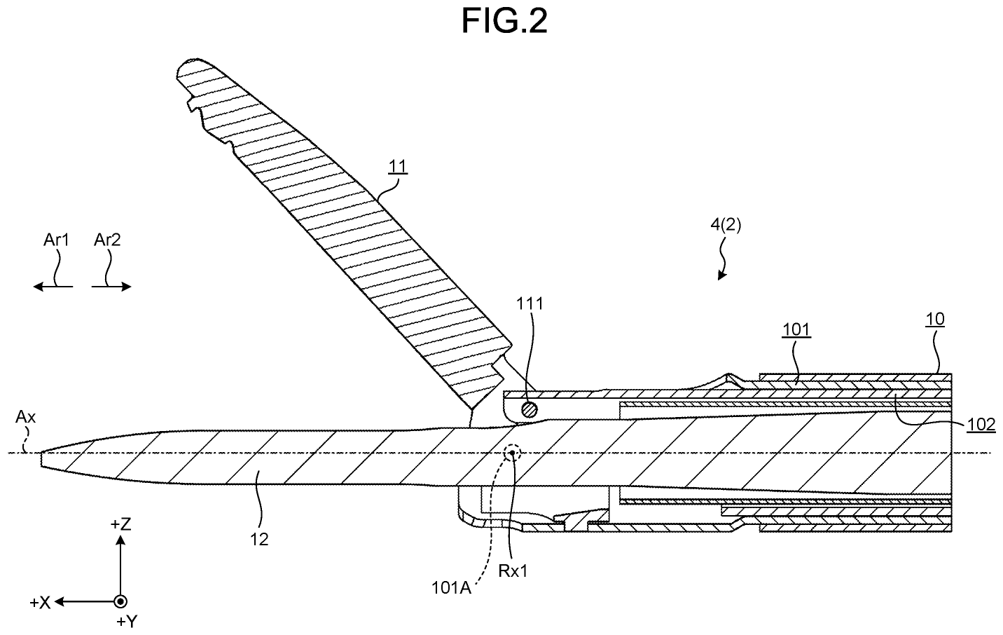

FIG. 2 is a diagram illustrating a portion on the distal end side Ar1 in the ultrasound treatment tool 2. Specifically, FIG. 2 is a cross-sectional view of a portion on the distal end side Ar1 of the ultrasound treatment tool 2, taken along an XZ plane including the center axis Ax.

The sheath 10 has substantially a cylindrical shape as a whole. As illustrated in FIG. 1 or 2, the sheath 10 includes an outer pipe 101, an inner pipe 102, a probe holder 103 (FIG. 1), a slider receiver 104 (FIG. 1), and the slider 105 (FIG. 1).

The outer pipe 101 is a cylindrical pipe.

The outer pipe 101 has an end on the distal end side Ar1 to which a first pin 101A (FIG. 2) is fixed, the first pin 101A extending in a direction along the Y-axis and pivotally supporting the jaw 11 so as to be rotatable about a rotation axis Rx1 (FIG. 2).

The inner pipe 102 is a cylindrical pipe having a smaller diameter dimension than the outer pipe 101. Furthermore, the inner pipe 102 is inserted into the outer pipe 101 so as to be coaxial with the outer pipe 101.

The inner pipe 102 has an end on the distal end side Ar1 into which a second pin 111 is inserted, the second pin 111 being provided at the jaw 11 and extending in parallel with the rotation axis Rx1 (first pin 101A).

The probe holder 103 has substantially a cylindrical shape, and is inserted into the rotation knob 9 and the holding case body 61 and held across the rotation knob 9 and the holding case body 61, as illustrated in FIG. 1. The probe holder 103 holds the vibration transmission member 12 inserted therein. In addition, the probe holder 103 has an end on the distal end side Ar1 where the probe holder 103 is mechanically connected to the rotation knob 9 and the outer pipe 101. In other words, the probe holder 103, the outer pipe 101, the jaw 11, and the vibration transmission member 12 rotate about the center axis Ax together with the rotation knob 9, in response to the rotation operation on the rotation knob 9 by the operator such as the surgeon.

The slider receiver 104 has substantially a cylindrical shape, and is arranged so as to be movable along the center axis Ax relative to the probe holder 103 being inserted therein. Here, the slider receiver 104 has an end on the distal end side Ar1 that is fixed to an end on the proximal end side Ar2 of the inner pipe 102, while being allowed to move along the center axis Ax relative to the probe holder 103 but while being restricted in rotation about the center axis Ax. In other words, the slider receiver 104 and the inner pipe 102 rotate about the center axis Ax together with the rotation knob 9, in response to the rotation operation on the rotation knob 9 by the operator such as the surgeon.

The slider 105 has substantially a cylindrical shape, and is arranged so as to be movable along the center axis Ax relative to the slider receiver 104 being inserted therein. As described above, the slider 105 is engaged with the movable handle 7.

The slider 105, the slider receiver 104, and the inner pipe 102 operate as described below, in response to the operation on the movable handle 7 by the operator such as the surgeon.

In response to the closing operation on the movable handle 7 by the operator such as the surgeon, the slider 105 engaged with the movable handle 7 is pressed to the distal end side Ar1 along the center axis Ax. Furthermore, the slider receiver 104 receives the pressing force from the slider 105 toward the distal end side Ar1 via a coil spring 106 (FIG. 1) disposed between the slider receiver 104 and the slider 105. Furthermore, the inner pipe 102 moves to the distal end side Ar1 along the center axis Ax in conjunction with the slider receiver 104, and pushes the second pin 111 toward the distal end side Ar1. Then, the jaw 11 rotates counterclockwise in FIG. 2 about the rotation axis Rx1. In other words, the jaw 11 moves in a direction (closing direction) approaching an end on the distal end side Ar1 of the vibration transmission member 12.

Furthermore, in response to the opening operation on the movable handle 7 by the operator such as the surgeon, the jaw 11 rotates clockwise in FIG. 2 about the rotation axis Rx1. In other words, the jaw 11 moves in a direction (opening direction) away from the end on the distal end side Ar1 of the vibration transmission member 12.

As described above, the jaw 11 is opened/closed with respect to the end on the distal end side Ar1 of the vibration transmission member 12, according to the operation on the movable handle 7 by the operator such as the surgeon.

As described above, the jaw 11 is opened/closed with respect to the end on the distal end side Ar1 of the vibration transmission member 12 to grip the target portion between the jaw 11 and the end on the distal end side Ar1.

The vibration transmission member 12 corresponds to an end effector. The vibration transmission member 12 has an elongated shape extending linearly along the center axis Ax. Furthermore, as illustrated in FIG. 2, the vibration transmission member 12 is inserted into the sheath 10 (the inner pipe 102 and the probe holder 103) while the end on the distal end side Ar1 protrudes outward. At this time, an end on the proximal end side Ar2 of the vibration transmission member 12 is mechanically connected to the ultrasound transducer device 5, as illustrated in FIG. 1. In other words, the ultrasound transducer device 5 rotates about the center axis Ax together with the vibration transmission member 12, in response to the rotation operation on the rotation knob 9 by the operator such as the surgeon. Then, the vibration transmission member 12 transmits ultrasound vibration generated by the ultrasound transducer device 5, from the end on the proximal end side Ar2 to the end on the distal end side Ar1. In the present embodiment, the ultrasound vibration is longitudinal vibration that vibrates in a direction along the center axis Ax.

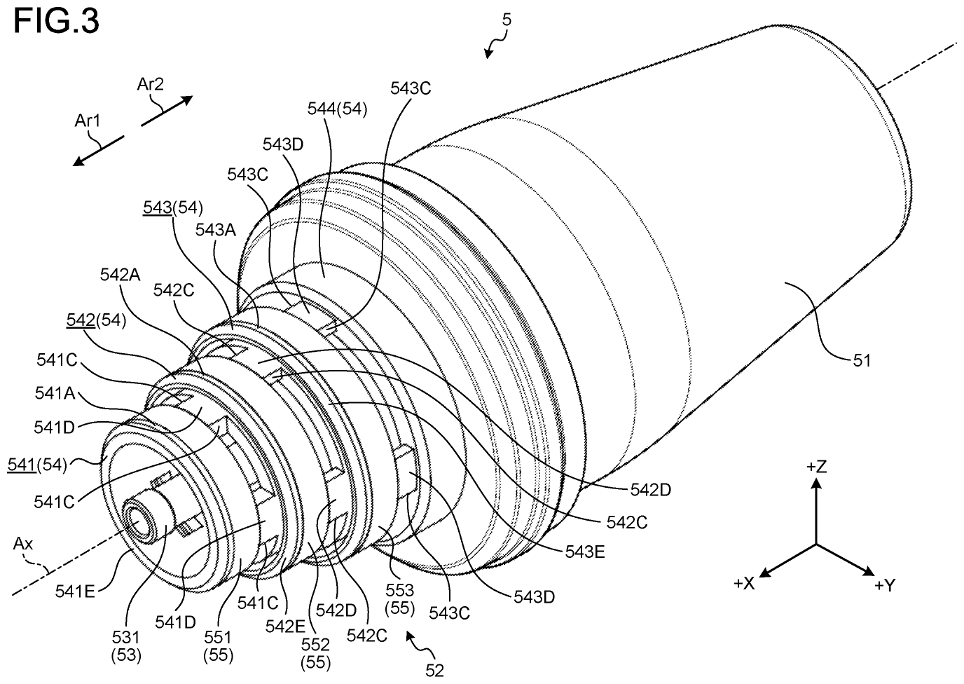

As illustrated in FIG. 1, the handpiece-side electrode unit 13 is fixed inside the holding case body 61. The handpiece-side electrode unit 13 has a function of electrically connecting the first to third wiring 82A to 82C connected to the circuit board 80 to a first electrode 55 (FIG. 3) provided at the ultrasound transducer device 5.

Note that a detailed configuration of the handpiece-side electrode unit 13 will be described in “Configuration of handpiece-side electrode unit” described later.

The ultrasound transducer device 5 is inserted into the holding case body 61 from the proximal end side Ar2 of the holding case body 61, and is configured to be detachable from the holding case body 61. Then, the ultrasound transducer device 5 is electrically connected to the control device 3 via an electric cable C (FIG. 1), and generates ultrasound vibration under the control of the control device 3.

Hereinafter, a detailed configuration of the ultrasound transducer device 5 will be described.

Configuration of Ultrasound Transducer Device

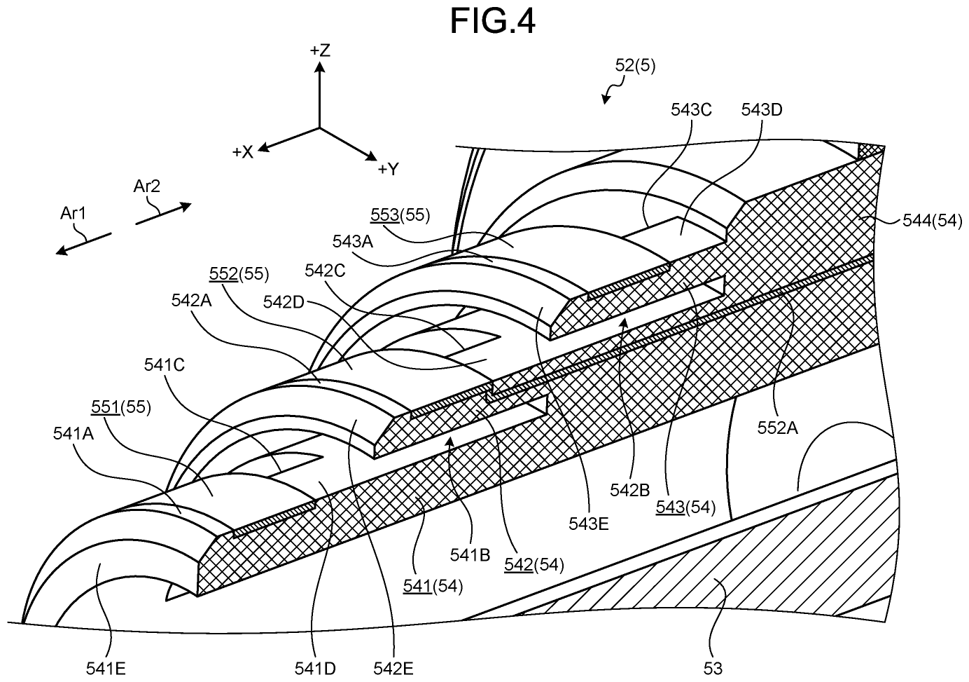

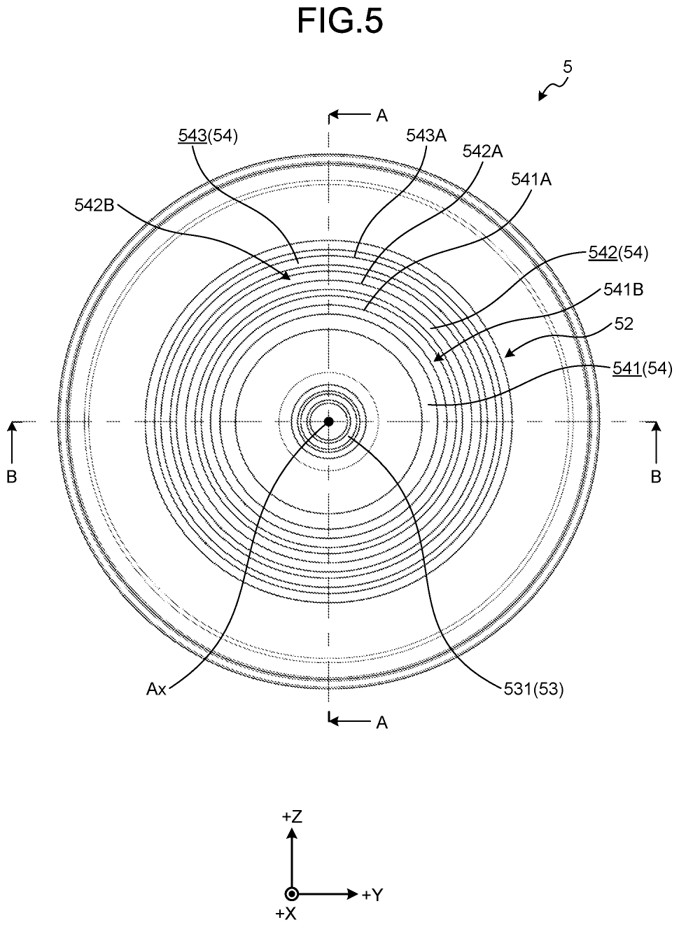

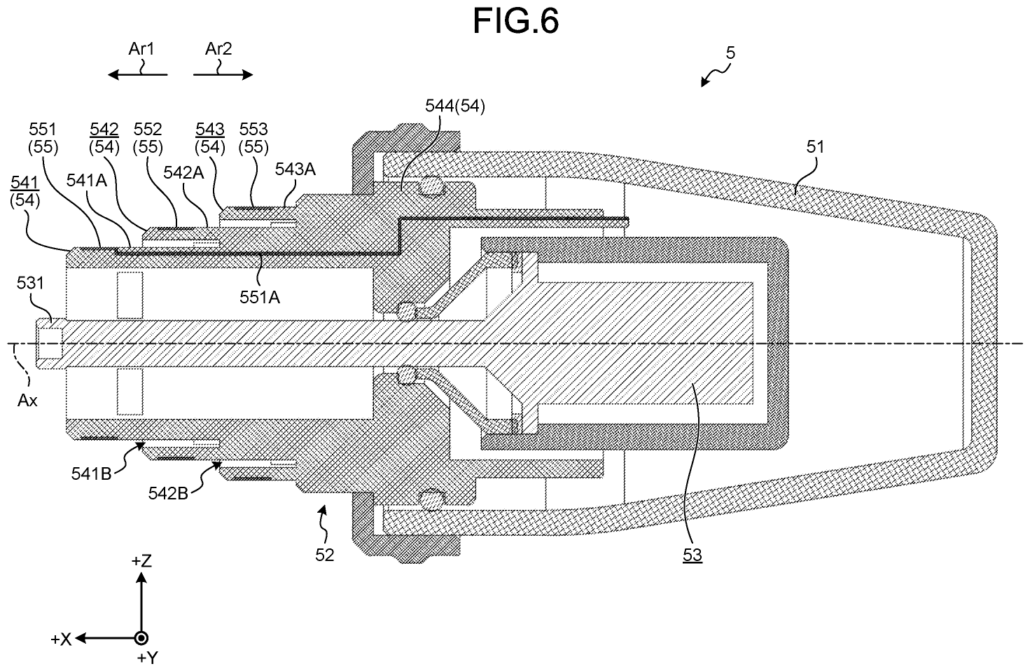

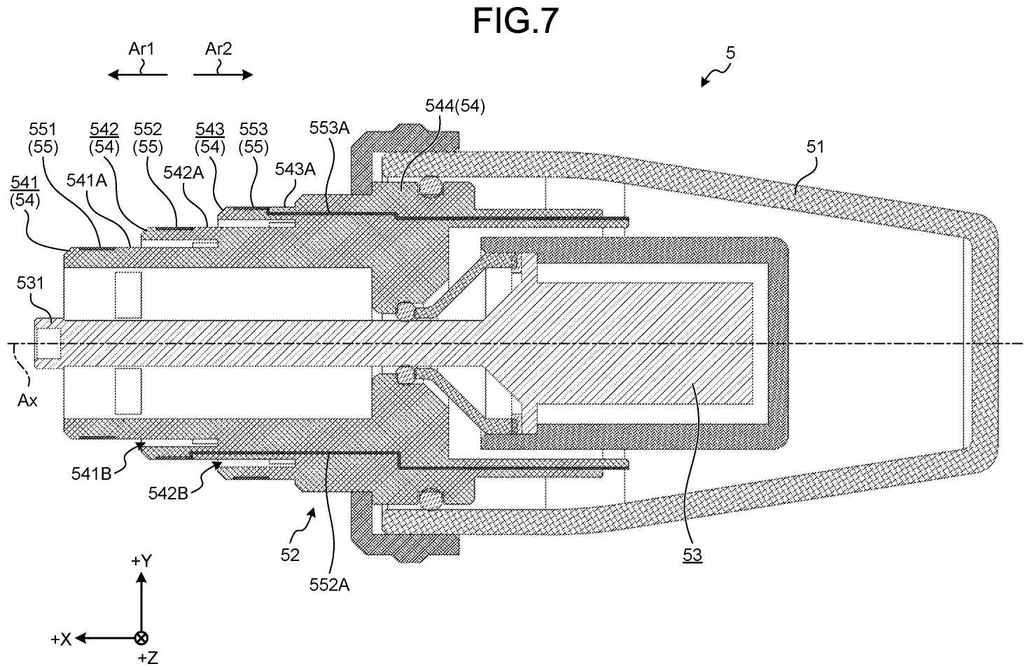

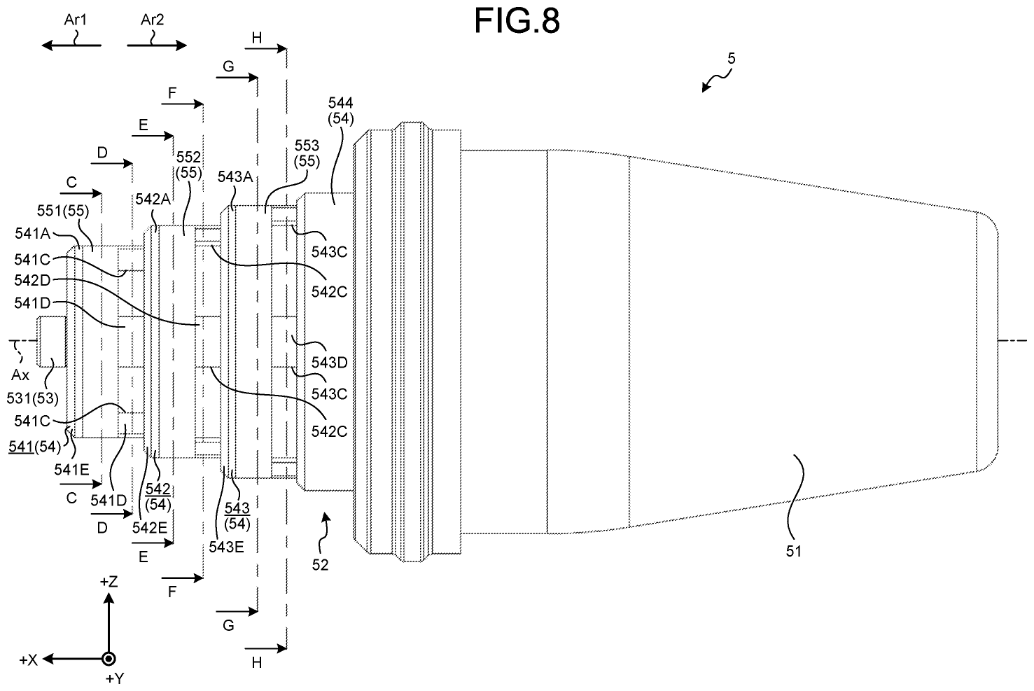

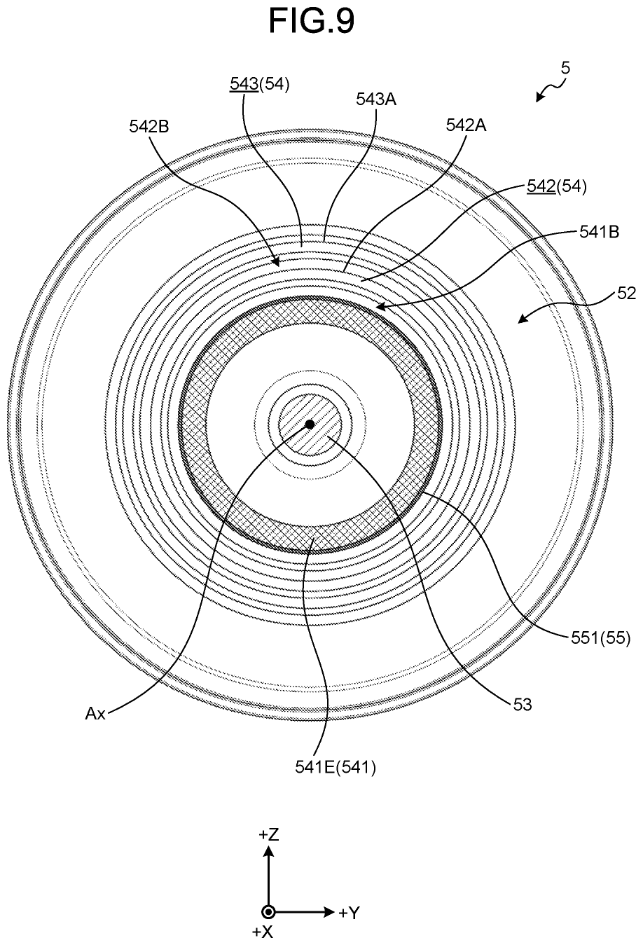

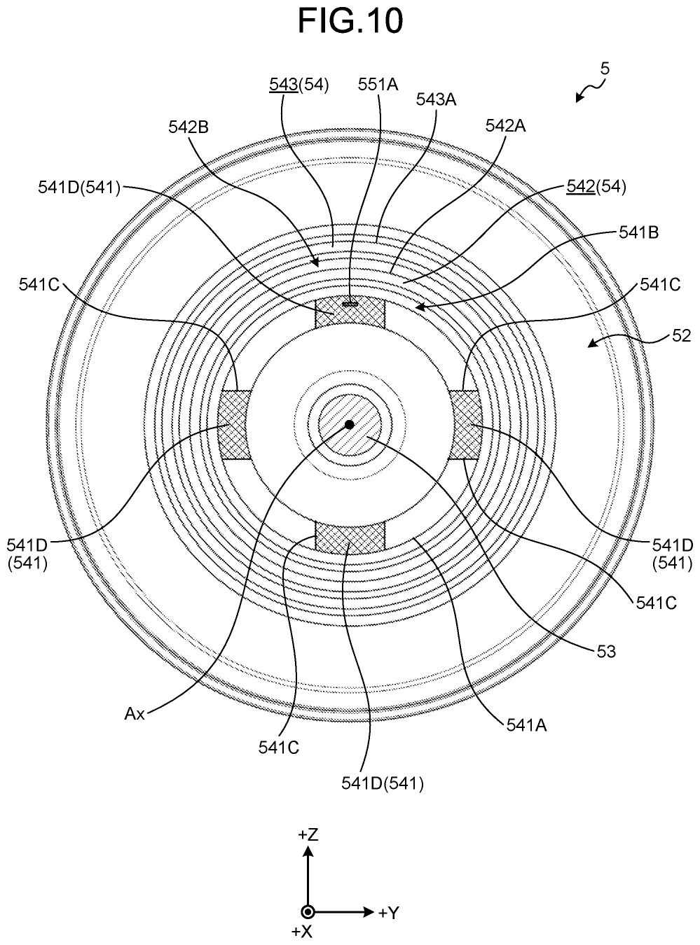

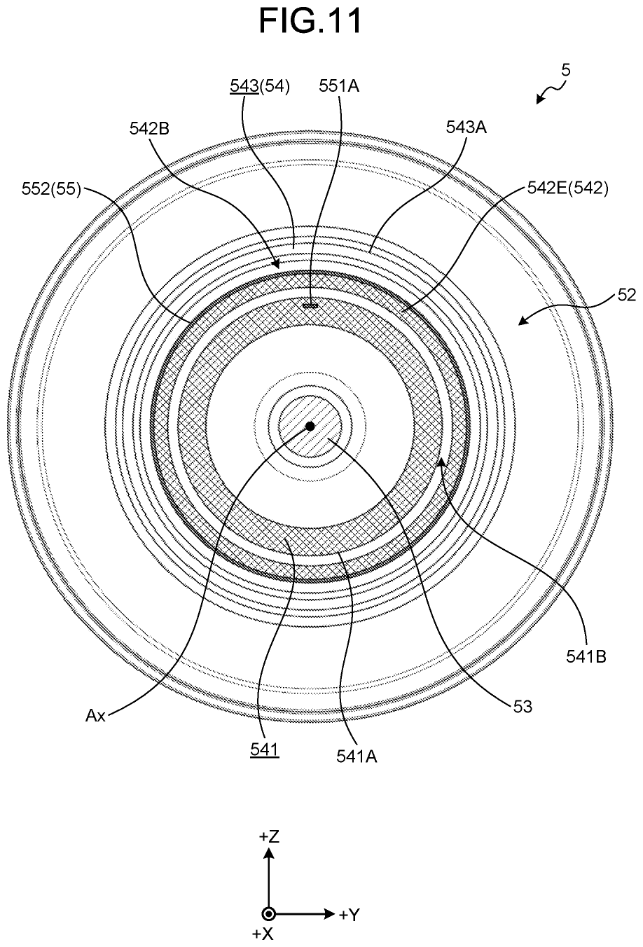

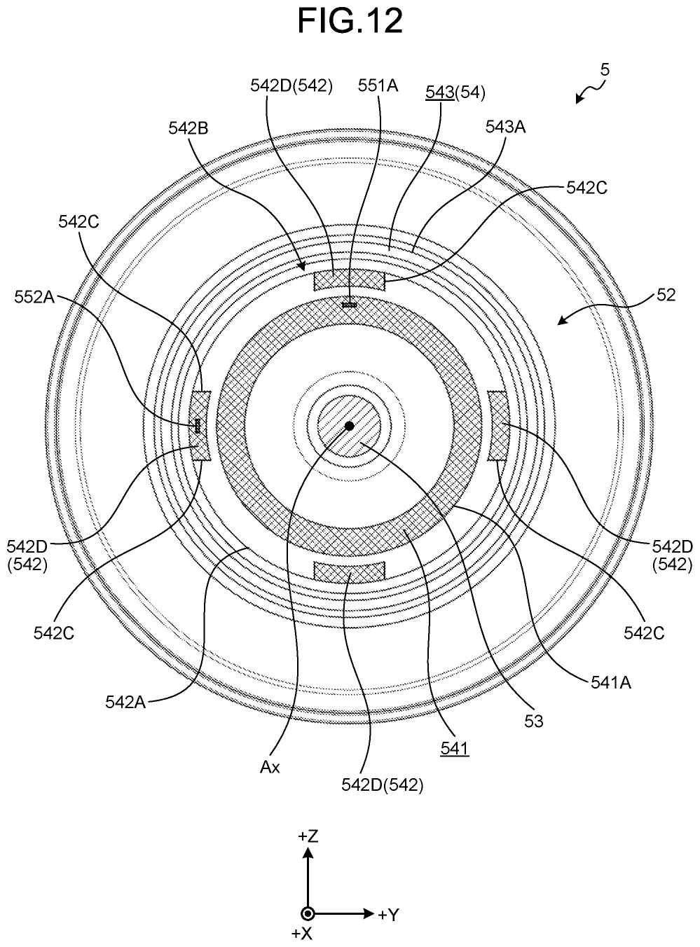

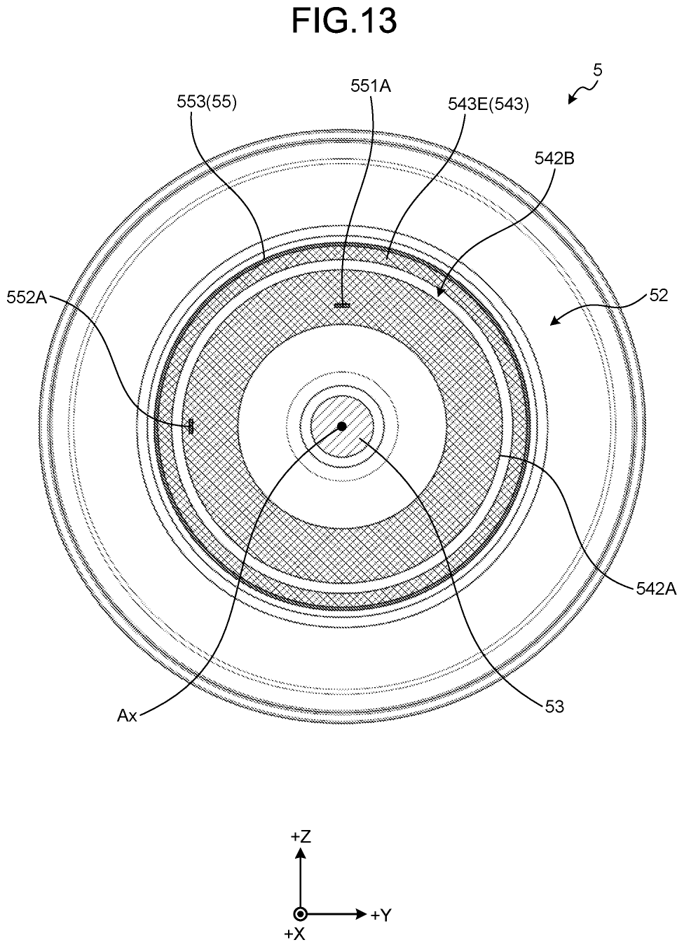

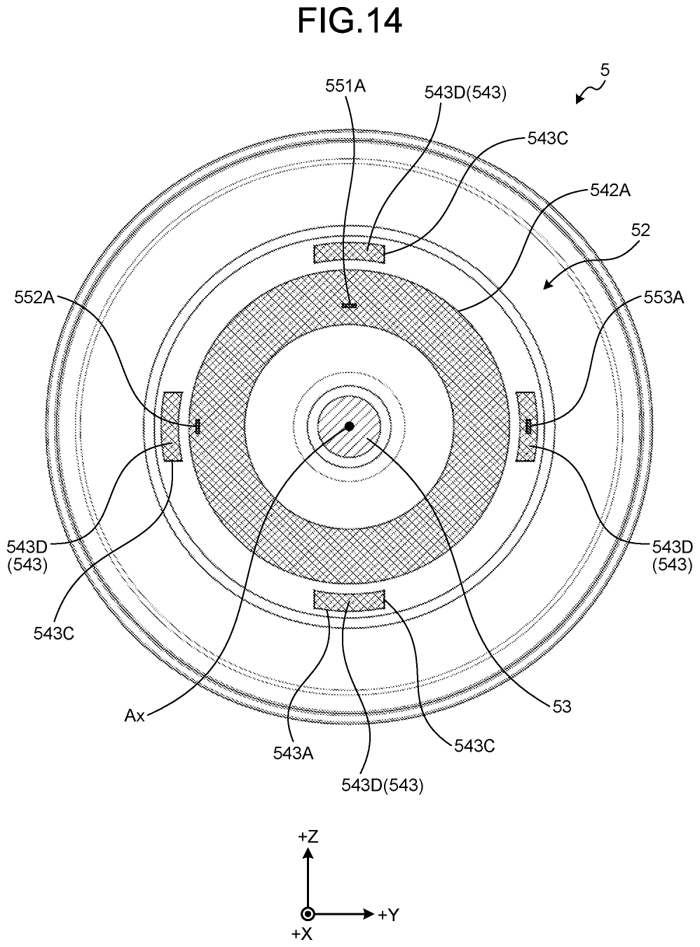

FIGS. 3 to 14 are diagrams illustrating the configuration of the ultrasound transducer device 5. Specifically, FIG. 3 is a perspective view of the ultrasound transducer device 5 as viewed from the distal end side Ar1. FIG. 4 is an enlarged view of a portion of a cross-section of a transducer (TD) side electrode unit 52 taken along an XZ plane including the center axis Ax. FIG. 5 is a diagram of the ultrasound transducer device 5 as viewed from the distal end side Ar1 along the center axis Ax. FIG. 6 is a cross-sectional view taken along the line A-A of FIG. 5. FIG. 7 is a cross-sectional view taken along the line B-B of FIG. 5. FIG. 8 is a diagram of the ultrasound transducer device 5 as viewed from the +Y axis side. FIG. 9 is a cross-sectional view taken along the line C-C of FIG. 8. FIG. 10 is a cross-sectional view taken along the line D-D of FIG. 8. FIG. 11 is a cross-sectional view taken along the line E-E of FIG. 8. FIG. 12 is a cross-sectional view taken along the line F-F of FIG. 8. FIG. 13 is a cross-sectional view taken along the line G-G of FIG. 8. FIG. 14 is a cross-sectional view taken along the line H-H of FIG. 8.

As illustrated in FIGS. 3 to 14, the ultrasound transducer device 5 includes a transducer (TD) case 51 (FIG. 3 and FIGS. 6 to 8), the TD-side electrode unit 52, and an ultrasound transducer 53.

As illustrated in FIG. 3 and FIGS. 6 to 8, the TD case 51 has a bottomed cylindrical shape in which the distal end side Ar1 opens. Furthermore, the electric cable C is routed from the outside to the inside of the TD case 51 through a side wall of the TD case 51 on the proximal end side Ar2.

As illustrated in FIGS. 3 to 14, the TD-side electrode unit 52 includes a first support member 54 and the first electrode 55.

As illustrated in FIG. 6 or 7, the first support member 54 is a cylindrical body that extends along the center axis Ax and is fitted into an opening portion of the TD case 51.

In the first support member 54, an outer surface of a portion protruding from the TD case 51 to the distal end side Ar1 is formed into a stepped shape having three steps 541A, 542A, and 543A (FIGS. 3 to 14) sequentially from the distal end side Ar1 toward the proximal end side Ar2. The three steps 541A, 542A, and 543A each have a circular cross-sectional shape about the center axis Ax and have outer diameter dimensions increasing in the order of the three steps 541A, 542A, and 543A.

Furthermore, the first support member 54 is provided with a first slit 541B (FIGS. 4 to 7 and FIGS. 9 to 11) that is formed by extending the step 541A toward the proximal end side Ar2, and a second slit 542B (FIGS. 4 to 7 and FIGS. 9 to 13) that is formed by extending the step 542A toward the proximal end side Ar2. The first and second slits 541B and 542B extend over the entire circumferential periphery around the center axis Ax, and correspond to spaces.

In the first support member 54, portions 541 to 543 (FIGS. 4 to 7 and FIGS. 9 to 11) are obtained by being divided into three divisions along the radial direction about the center axis Ax by the first and second slits 541B and 542B, and the portions 541 to 543 correspond to first deformation portions. Hereinafter, for convenience of description, the portion 541 is referred to as a first inner-peripheral-side deformation portion 541, the portion 542 as a first intermediate deformation portion 542, and the portion 543 as a first outer-peripheral-side deformation portion 543. More specifically, the first inner-peripheral-side deformation portion 541 is an annular portion having the step 541A as an outer peripheral surface. The first intermediate deformation portion 542 is an annular portion having the step 542A as an outer peripheral surface. The first outer-peripheral-side deformation portion 543 is an annular portion having the step 543A as an outer peripheral surface.

Furthermore, in the first support member 54, a cylindrical portion 544 (FIGS. 4, 6, and 7) is located nearer the proximal end side Ar2 than the deformation portions 541 to 543, and the cylindrical portion 544 corresponds to a support base. Hereinafter, for convenience of description, the portion 544 is referred to as a support base 544. In other words, each of the deformation portions 541 to 543 protrudes along the center axis Ax from an end surface of the support base 544 on the distal end side Ar1. Furthermore, in the deformation portions 541 to 543, a protrusion dimension from the support base 544 is set larger as the outer diameter dimension decreases. In other words, the first inner-peripheral-side deformation portion 541 is set to have a maximum length, and the first outer-peripheral-side deformation portion 543 is set to have a minimum length.

Furthermore, the first inner-peripheral-side deformation portion 541 is provided with four openings 541C (FIGS. 3, 4, 8, and 10) each penetrating from an outer peripheral surface (the step 541A) to an inner peripheral surface. The four openings 541C have the same size and are provided at positions rotationally symmetric positions around the center axis Ax by 90°. Hereinafter, for convenience of description, in the first inner-peripheral-side deformation portion 541, four portions adjacent to the openings 541C in the circumferential direction around the center axis Ax are referred to as arm portions 541D (FIGS. 3, 4, 8, and 10). Furthermore, in the first inner-peripheral-side deformation portion 541, an annular-shaped portion connected to ends on the distal end side Ar1 of the four arm portions 541D is referred to as an annular portion 541E (FIGS. 3, 4, 8, and 9).

Then, when an external force acts on the annular portion 541E, the four arm portions 541D are elastically deformed, and the position of the annular portion 541E is changed.

The first intermediate deformation portion 542 is provided with four openings 542C (FIGS. 3, 4, 8, and 12) each penetrating from an outer peripheral surface (the step 542A) to an inner peripheral surface (the first slit 541B). The four openings 542C have the same size and are provided at positions rotationally symmetric positions around the center axis Ax by 90°. Hereinafter, for convenience of description, in the first intermediate deformation portion 542, four portions adjacent to the openings 542C in the circumferential direction around the center axis Ax are referred to as arm portions 542D (FIGS. 3, 4, 8, and 12). Furthermore, in the first intermediate deformation portion 542, an annular-shaped portion connected to ends on the distal end side Ar1 of the four arm portions 542D is referred to as an annular portion 542E (FIGS. 3, 4, 8, and 11).

Then, when an external force acts on the annular portion 542E, the four arm portions 542D are elastically deformed, and the position of the annular portion 542E is changed.

The first outer-peripheral-side deformation portion 543 is provided with four openings 543C (FIGS. 3, 4, 8, and 14) each penetrating from an outer peripheral surface (the step 543A) to an inner peripheral surface (second slit 542B). The four openings 543C have the same size and are provided at positions rotationally symmetric positions around the center axis Ax by 90°. Hereinafter, for convenience of description, in the first outer-peripheral-side deformation portion 543, four portions adjacent to the openings 543C in the circumferential direction around the center axis Ax are referred to as arm portions 543D (FIGS. 3, 4, 8, and 14). Furthermore, in the first outer-peripheral-side deformation portion 543, an annular-shaped portion connected to ends on the distal end side Ar1 of the four arm portions 543D is referred to as an annular portion 543E (FIGS. 3, 4, 8, and 13).

Then, when an external force acts on the annular portion 543E, the four arm portions 543D are elastically deformed, and the position of the annular portion 543E is changed.

Note that the arm portions 541D, 542D, and 543D have configurations independent of each other. Therefore, when the external force acts on the respective annular portions 541E, 542E, and 543E, the positions of the annular portions 541E, 542E, and 543E are changed independently of each other.

Furthermore, the number of the openings 541C is not limited to four, and five or more or three or less openings 541C may be provided, or a configuration with no opening may be adopted. The same applies to the openings 542C and 543C.

The number of the first electrodes 55 (three first electrodes 55 in the present embodiment) is the same as the number of deformation portions 541 to 543, and the first electrodes 55 are supported by the deformation portions 541 to 543. Hereinafter, for convenience of description, the first electrode 55 supported by the first inner-peripheral-side deformation portion 541 is referred to as a first inner-peripheral-side electrode 551 (FIGS. 3 and 4, and FIGS. 6 to 9), the first electrode 55 supported by the first intermediate deformation portion 542 is referred to as a first intermediate electrode 552 (FIGS. 3 and 4, FIGS. 6 to 8, and FIG. 11), and the first electrode 55 supported by the first outer-peripheral-side deformation portion 543 is referred to as a first outer-peripheral-side electrode 553 (FIGS. 3 and 4, FIGS. 6 to 8, and FIG. 13).

The first inner-peripheral-side electrode 551 is made of a conductive material and has an annular shape surrounding the center axis Ax. The first inner-peripheral-side electrode 551 is provided on an outer peripheral surface (the step 541A) of the annular portion 541E and formed by, for example, insert molding. In other words, the first inner-peripheral-side electrode 551 has an outer diameter dimension that is substantially the same as the annular portion 541E.

Furthermore, the first support member 54 is provided with inner-peripheral-side wiring 551A (FIG. 6, FIGS. 10 to 14) that is electrically connected to the first inner-peripheral-side electrode 551 and extends from a connection position with the first inner-peripheral-side electrode 551 toward the proximal end side Ar2. The inner-peripheral-side wiring 551A extends inside the first inner-peripheral-side deformation portion 541 and support base 544 so as not be exposed from the outer peripheral surfaces and the inner peripheral surfaces of the first inner-peripheral-side deformation portion 541 and support base 544 (FIG. 6, FIGS. 10 to 14). Furthermore, part of the inner-peripheral-side wiring 551A extends inside each of the arm portions 541D (FIG. 10).

Although not specifically illustrated, the inner-peripheral-side wiring 551A is electrically connected to the electric cable C, in the TD case 51.

The first intermediate electrode 552 is made of a conductive material and has an annular shape surrounding the center axis Ax. The first intermediate electrode 552 is provided on an outer peripheral surface (the step 542A) of the annular portion 542E and formed by, for example, insert molding. In other words, the first intermediate electrode 552 has an outer diameter dimension that is substantially the same as the annular portion 542E.

Furthermore, the first support member 54 is provided with intermediate wiring 552A (FIGS. 4 and 7, FIGS. 12 to 14) that is electrically connected to the first intermediate electrode 552 and extends from a connection position with the first intermediate electrode 552 toward the proximal end side Ar2. The intermediate wiring 552A extends inside the first intermediate deformation portion 542 and support base 544 so as not be exposed from the outer peripheral surfaces and the inner peripheral surfaces of the first intermediate deformation portion 542 and support base 544 (FIGS. 4 and 7, FIGS. 12 to 14). Furthermore, part of the intermediate wiring 552A extends inside each of the arm portions 542D (FIG. 12).

Although not specifically illustrated, the intermediate wiring 552A is electrically connected to the electric cable C, in the TD case 51.

The first outer-peripheral-side electrode 553 is made of a conductive material and has an annular shape surrounding the center axis Ax. The first outer-peripheral-side electrode 553 is provided on an outer peripheral surface (the step 543A) of the annular portion 543E and formed by, for example, insert molding. In other words, the first outer-peripheral-side electrode 553 has an outer diameter dimension that is substantially the same as the annular portion 543E.

Furthermore, the first support member 54 is provided with outer-peripheral-side wiring 553A (FIGS. 7 and 14) that is electrically connected to the first outer-peripheral-side electrode 553 and extends from a connection position with the first outer-peripheral-side electrode 553 toward the proximal end side Ar2. The outer-peripheral-side wiring 553A extends inside the first outer-peripheral-side deformation portion 543 and the support base 544 so as not be exposed from the outer peripheral surfaces and the inner peripheral surfaces of the first outer-peripheral-side deformation portion 543 and support base 544 (FIGS. 7 and 14). Furthermore, part of the outer-peripheral-side wiring 553A extends inside each of the arm portions 543D (FIG. 14).

Although not specifically illustrated, the outer-peripheral-side wiring 553A is electrically connected to the electric cable C, in the TD case 51.

As illustrated in FIG. 6 or 7, the ultrasound transducer 53 is inserted into the TD case 51 and the first support member 54 and held across the TD case 51 and the first support member 54, and supported on the inner peripheral surface of the first support member 54. Then, the ultrasound transducer 53 is electrically connected to the electric cable C inside the TD case 51, and generates ultrasound vibration according to a drive signal output from the control device 3 via the electric cable C. In the present embodiment, the ultrasound transducer 53 includes a bolt-clamped Langevin type transducer (BLT).

In the ultrasound transducer 53, a probe mount portion 531 (FIG. 3, FIGS. 5 to 8) is provided at an end on the distal end side Ar1. When the ultrasound transducer device 5 is connected to the holding case body 61, the probe mount portion 531 is mechanically connected to an end on the proximal end side Ar2 of the vibration transmission member 12. This configuration causes rotation of the ultrasound transducer device 5 about the center axis Ax together with the vibration transmission member 12 in response to the rotation operation on the rotation knob 9 by the operator such as the surgeon.

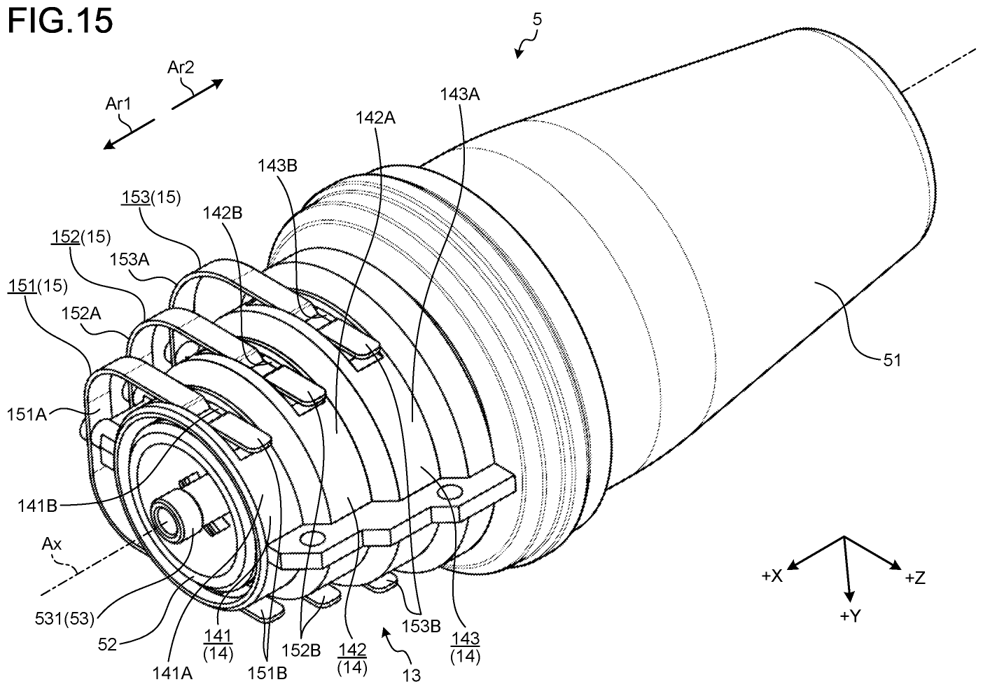

Configuration of Handpiece-side Electrode Unit Next, a detailed configuration of the handpiece-side electrode unit 13 will be described with reference to FIGS. 15 to 22.

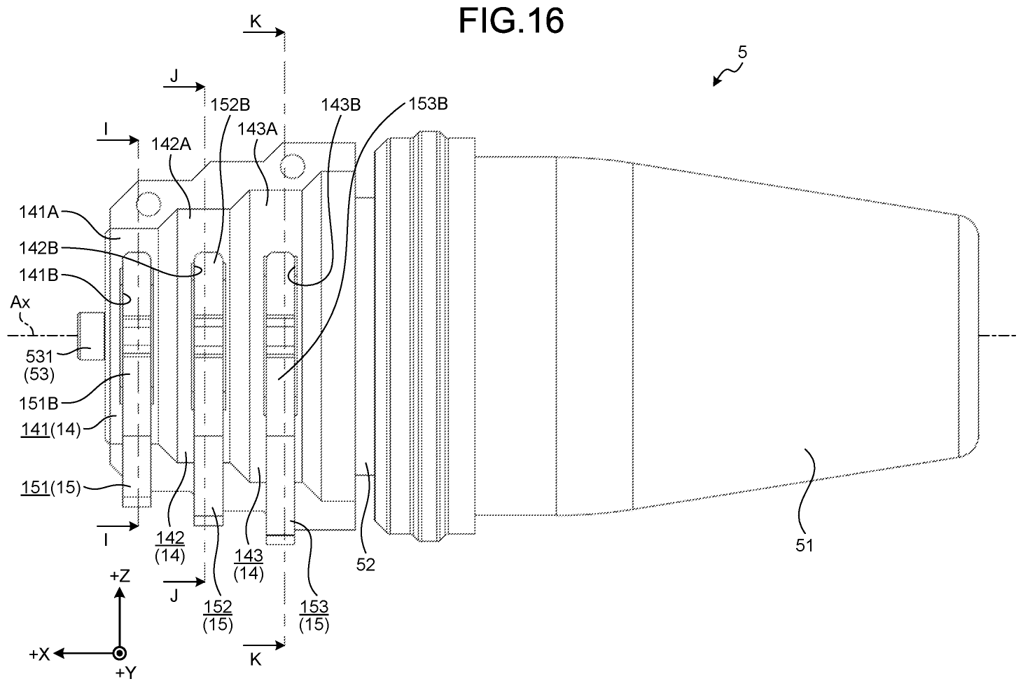

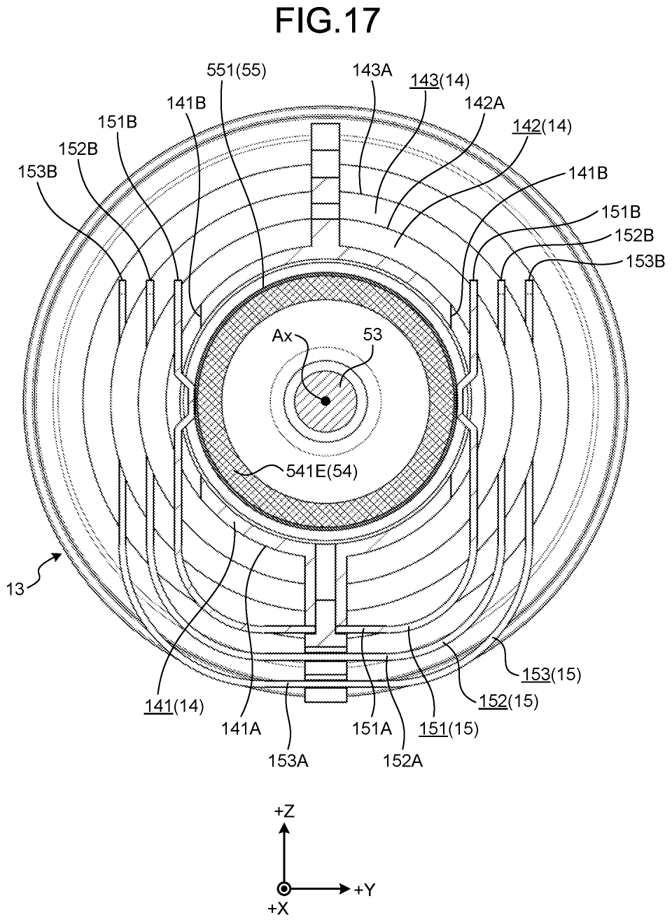

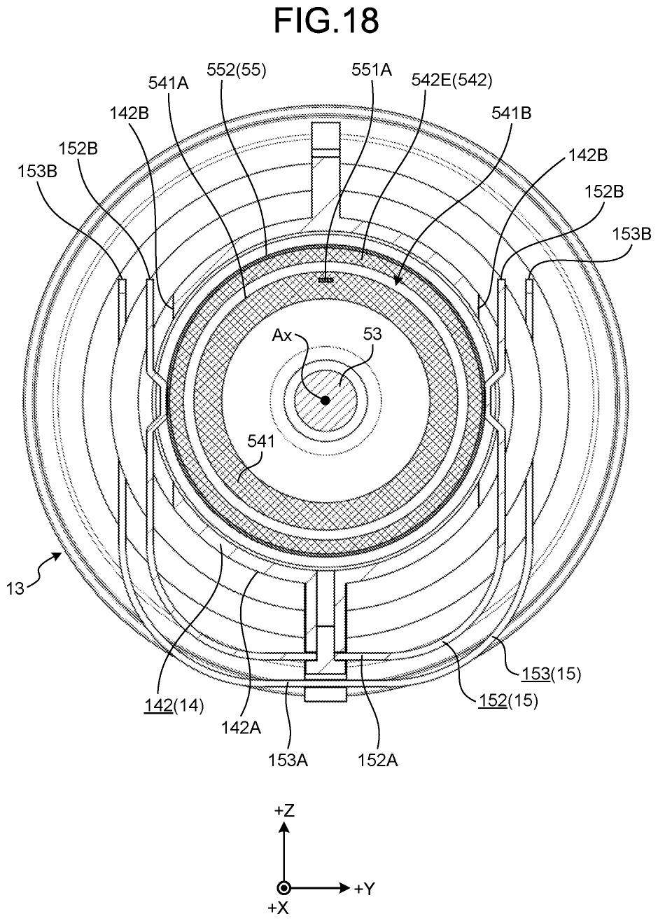

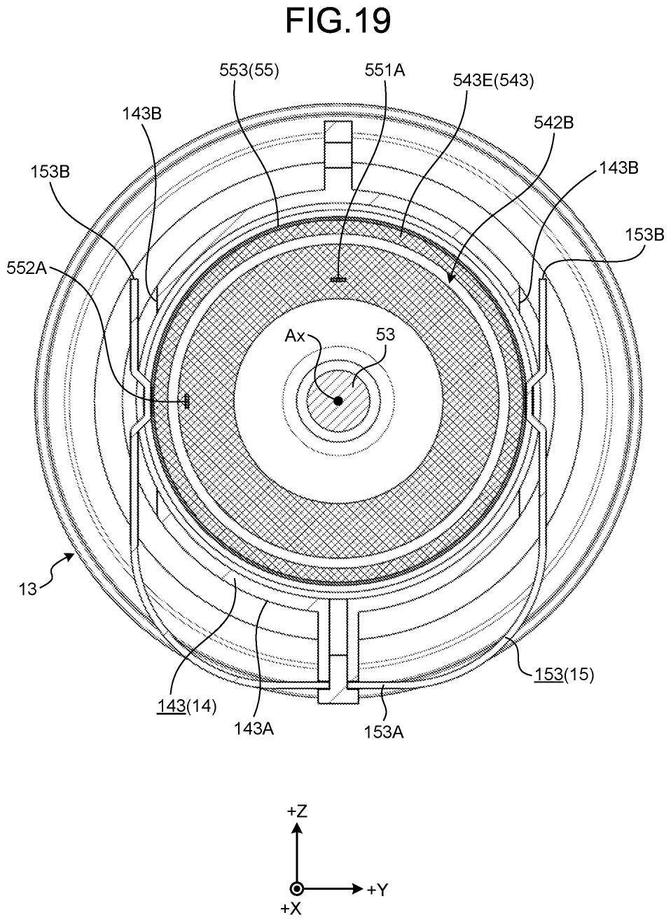

FIGS. 15 to 19 are diagrams illustrating the configuration of the handpiece-side electrode unit 13. Specifically, FIG. 15 is a perspective view illustrating a state in which the handpiece-side electrode unit 13 is assembled to the ultrasound transducer device 5 illustrated in FIG. 3. FIG. 16 is a diagram illustrating a state in which the handpiece-side electrode unit 13 is assembled to the ultrasound transducer device 5 illustrated in FIG. 8. FIG. 17 is a cross-sectional view taken along the line I-I of FIG. 16. Note that FIG. 17 is a cross-sectional view taken along the same plane as that of FIG. 9. FIG. 18 is a cross-sectional view taken along the line J-J of FIG. 16. Note that FIG. 18 is a cross-sectional view taken along the same plane as that of FIG. 11. FIG. 19 is a cross-sectional view taken along the line K-K of FIG. 16. Note that FIG. 19 is a cross-sectional view taken along the same plane as that of FIG. 13.

As illustrated in FIGS. 15 to 19, the handpiece-side electrode unit 13 includes a second support member 14 and a second electrode 15.

As illustrated in FIGS. 15 to 19, the second support member 14 is a cylindrical body that extends along the center axis Ax and is fixed inside the holding case body 61. When the ultrasound transducer device 5 is connected to the holding case body 61, the TD-side electrode unit 52 is inserted into the second support member 14.

As illustrated in FIG. 15 or 16, an outer surface of the second support member 14 is formed in a stepped shape having three steps 141A, 142A, and 143A sequentially from the distal end side Ar1 toward the proximal end side Ar2. The three steps 141A, 142A, and 143A each have a circular cross-sectional shape about the center axis Ax and have outer diameter dimensions increasing in the order of the three steps 141A, 142A, and 143A.

In the second support member 14, an annular portion 141 having the step 141A as an outer peripheral surface, an annular portion 142 having the step 142A as an outer peripheral surface, and an annular portion 143 having the step 143A as an outer peripheral surface correspond to a second deformation portion. Hereinafter, for convenience of description, the portion 141 is referred to as a second inner-peripheral-side deformation portion 141, the portion 142 is referred to as a second intermediate deformation portion 142, and the portion 143 is referred to as a second outer-peripheral-side deformation portion 143.

The second inner-peripheral-side deformation portion 141 has an inner diameter dimension that is set slightly larger than the outer diameter dimension of the first inner-peripheral-side deformation portion 541. When the ultrasound transducer device 5 is connected to the holding case body 61, an inner peripheral surface of the second inner-peripheral-side deformation portion 141 is opposed to the outer peripheral surface of the first inner-peripheral-side deformation portion 541 (FIG. 17).

The second inner-peripheral-side deformation portion 141 is provided with a pair of openings 141B (FIG. 17) that penetrate the inside and outside of the second inner-peripheral-side deformation portion 141 along the Y-axis.

The second intermediate deformation portion 142 has an inner diameter dimension that is set slightly larger than the outer diameter dimension of the first intermediate deformation portion 542. When the ultrasound transducer device 5 is connected to the holding case body 61, an inner peripheral surface of the second intermediate deformation portion 142 is opposed to the outer peripheral surface of the first intermediate deformation portion 542 (FIG. 18).

The second intermediate deformation portion 142 is provided with a pair of openings 142B (FIG. 18) that penetrate the inside and outside of the second intermediate deformation portion 142 along the Y-axis.

The second outer-peripheral-side deformation portion 143 has an inner diameter dimension that is set slightly larger than the outer diameter dimension of the first outer-peripheral-side deformation portion 543. When the ultrasound transducer device 5 is connected to the holding case body 61, an inner peripheral surface of the second outer-peripheral-side deformation portion 143 is opposed to the outer peripheral surface of the first outer-peripheral-side deformation portion 543 (FIG. 19).

The second outer-peripheral-side deformation portion 143 is provided with a pair of openings 143B (FIG. 19) that penetrate the inside and outside of the second outer-peripheral-side deformation portion 143 along the Y-axis.

The number of the second electrodes 15 (three second electrodes 15 in the present embodiment) is the same as the number of deformation portions 141 to 143, and the second electrodes 15 are supported by the deformation portions 141 to 143. Hereinafter, for convenience of description, the second electrode 15 supported by the second inner-peripheral-side deformation portion 141 is referred to as a second inner-peripheral-side electrode 151 (FIGS. 15 to 17), the second electrode 15 supported by the second intermediate deformation portion 142 is referred to as a second intermediate electrode 152 (FIGS. 15 to 18), and the second electrode 15 supported by the second outer-peripheral-side deformation portion 143 is referred to as a second outer-peripheral-side electrode 153 (FIGS. 15 to 19).

The second inner-peripheral-side electrode 151 is made of a conductive material. As illustrated in FIGS. 15 to 17, the second inner-peripheral-side electrode 151 includes an electrode base portion 151A and a pair of leaf spring portions 151B, and has substantially a U-shape as a whole.

The electrode base portion 151A has a flat plate shape extending along the Y-axis, and has plate surfaces each of which is a portion fixed to an outer peripheral surface of the second inner-peripheral-side deformation portion 141 in a posture orthogonal to the Z-axis. In addition, as illustrated in FIG. 1, the first wiring 82A is electrically connected to the electrode base portion 151A by soldering or the like.

The pair of leaf spring portions 151B are portions that extend in a +Z-axis direction from both ends of the electrode base portion 151A, and the portions are configured to be elastically deformable in Y-axis directions with both ends as fulcrums. Furthermore, in a state where the electrode base portion 151A is fixed to the outer peripheral surface of the second inner-peripheral-side deformation portion 141, the pair of leaf spring portions 151B are partially exposed to the inside of the second inner-peripheral-side deformation portion 141 through the pair of openings 141B. When the ultrasound transducer device 5 is connected to the holding case body 61, the second inner-peripheral-side electrode 151 (the pair of leaf spring portions 151B) abuts on the first inner-peripheral-side electrode 551 and is electrically connected to the first inner-peripheral-side electrode 551 (FIG. 17). Note that, the first inner-peripheral-side electrode 551 has the annular shape, and thus, the first inner-peripheral-side electrode 551 is always electrically connected to the second inner-peripheral-side electrode 151, even if rotating about the center axis Ax relative to the second inner-peripheral-side electrode 151 in response to the rotation operation on the rotation knob 9 by the operator such as the surgeon. Then, the first wiring 82A is electrically connected to the control device 3 through a first electric path from the second inner-peripheral-side electrode 151 to the electric cable C through the first inner-peripheral-side electrode 551 and the inner-peripheral-side wiring 551A.

The second intermediate electrode 152 is made of a conductive material. As illustrated in FIGS. 15 to 18, the second intermediate electrode 152 includes an electrode base portion 152A and a pair of leaf spring portions 152B, and has a substantially U-shape as a whole.

The electrode base portion 152A has a flat plate shape that has a longitudinal length larger than that of the electrode base portion 151A, corresponding to the outer diameter dimension of the second intermediate deformation portion 142. Then, the electrode base portion 152A is fixed to an outer peripheral surface of the second intermediate deformation portion 142 with each plate surface in a posture orthogonal to the Z-axis. In addition, as illustrated in FIG. 1, the second wiring 82B is electrically connected to the electrode base portion 152A by soldering or the like.

The pair of leaf spring portions 152B are portions that extend in a +Z-axis direction from both ends of the electrode base portion 152A, and the portions are configured to be elastically deformable in Y-axis directions with both ends as fulcrums. Each of the pair of leaf spring portions 152B has the same shape as the corresponding leaf spring portion 151B. Furthermore, in a state where the electrode base portion 152A is fixed to the outer peripheral surface of the second intermediate deformation portion 142, the pair of leaf spring portions 152B are partially exposed to the inside of the second intermediate deformation portion 142 through the pair of openings 142B. When the ultrasound transducer device 5 is connected to the holding case body 61, the second intermediate electrode 152 (the pair of leaf spring portions 152B) abuts on the first intermediate electrode 552 and is electrically connected to the first intermediate electrode 552 (FIG. 18). Note that, the first intermediate electrode 552 has the annular shape, and thus, the first intermediate electrode 552 is always electrically connected to the second intermediate electrode 152, even if rotating about the center axis Ax relative to the second intermediate electrode 152 in response to the rotation operation on the rotation knob 9 by the operator such as the surgeon. Then, the second wiring 82B is electrically connected to the control device 3 through a second electric path from the second intermediate electrode 152 to the electric cable C through the first intermediate electrode 552 and the intermediate wiring 552A.

The second outer-peripheral-side electrode 153 is made of a conductive material. As illustrated in FIGS. 15 to 19, the second outer-peripheral-side electrode 153 includes an electrode base portion 153A and a pair of leaf spring portions 153B, and has a substantially U-shape as a whole.

The electrode base portion 153A has a flat plate shape that has a longitudinal length larger than that of the electrode base portion 152A corresponding to the outer diameter dimension of the second outer-peripheral-side deformation portion 143. Then, the electrode base portion 153A is fixed to an outer peripheral surface of the second outer-peripheral-side deformation portion 143 with each plate surface in a posture orthogonal to the Z-axis. In addition, as illustrated in FIG. 1, the third wiring 82C is electrically connected to the electrode base portion 153A by soldering or the like.

The pair of leaf spring portions 153B are portions that extend in a +Z-axis direction from both ends of the electrode base portion 153A, and the portions are configured to be elastically deformable in Y-axis directions with both ends as fulcrums. Each of the pair of leaf spring portions 153B has the same shape as the corresponding leaf spring portion 151B. Furthermore, in a state where the electrode base portion 153A is fixed to the outer peripheral surface of the second outer-peripheral-side deformation portion 143, the pair of leaf spring portions 153B are partially exposed to the inside of the second outer-peripheral-side deformation portion 143 through the pair of openings 143B. When the ultrasound transducer device 5 is connected to the holding case body 61, the second outer-peripheral-side electrode 153 (the pair of leaf spring portions 153B) abuts on the first outer-peripheral-side electrode 553 and is electrically connected to the first outer-peripheral-side electrode 553 (FIG. 19). Note that, the first outer-peripheral-side electrode 553 has the annular shape, and thus, the first outer-peripheral-side electrode 553 is always electrically connected to the second outer-peripheral-side electrode 153, even if rotating about the center axis Ax relative to the second outer-peripheral-side electrode 153 in response to the rotation operation on the rotation knob 9 by the operator such as the surgeon. Then, the third wiring 82C is electrically connected to the control device 3 through a third electric path from the second outer-peripheral-side electrode 153 to the electric cable C through the first outer-peripheral-side electrode 553 and the outer-peripheral-side wiring 553A.

Configuration of Control Device

The control device 3 integrally controls the operations of the ultrasound treatment tool 2.

Specifically, the control device 3 uses the first to third electric paths described above to determine whether the setting operation for the first energy output mode or the second energy output mode has been performed by the operator such as the surgeon.

Then, when it is determined that the setting operation for the first energy output mode has been performed, the control device 3 outputs the drive signal according to the first energy output mode to the ultrasound transducer 53 through the electric cable C. The ultrasound transducer 53 thereby generates ultrasound vibration. Then, the ultrasound vibration is applied from the end on the distal end side Ar1 of the vibration transmission member 12 to the target portion gripped between the jaw 11 and the end on the distal end side Ar1, for performing the treatment corresponding to the first energy output mode.

Then, when it is determined that the setting operation for the second energy output mode has been performed, the control device 3 outputs the drive signal according to the second energy output mode to the ultrasound transducer 53 through the electric cable C. Therefore, the treatment corresponding to the second energy output mode is performed on the target portion gripped between the jaw 11 and the end on the distal end side Ar1 of the vibration transmission member 12.

According to the present embodiment described above, the following effects are obtained.

In the present embodiment, when an external force acts on the annular portion 541E, the four arm portions 541D are elastically deformed, and the position of the annular portion 541E is changed. In other words, the first inner-peripheral-side deformation portion 541 is elastically deformed according to the external force, thereby moving the first inner-peripheral-side electrode 551. Furthermore, when an external force acts on the annular portion 542E, the four arm portions 542D are elastically deformed, and the position of the annular portion 542E is changed. In other words, the first intermediate deformation portion 542 is elastically deformed according to the external force, thereby moving the first intermediate electrode 552. Furthermore, when an external force acts on the annular portion 543E, the four arm portions 543D are elastically deformed, and the position of the annular portion 543E is changed. In other words, the first outer-peripheral-side deformation portion 543 is elastically deformed according to the external force, thereby moving the first outer-peripheral-side electrode 553.

Therefore, even when the first support member 54 and the second support member 14 are manufactured with dimensions different from design dimensions due to a manufacturing error, and contact pressure between the first electrode 55 and the second electrode 15 becomes larger than design contact pressure, the positions of the first electrodes 55 are movable due to the deformation portions 541 to 543, and thus, the contact pressure can be reduced. Therefore, even when the ultrasound transducer device 5 rotates together with the vibration transmission member 12 in response to the rotation operation on the rotation knob 9 by the operator such as the surgeon, the contact pressure between the first electrode 55 and the second electrode 15 is reduced, thus reducing wear between the first electrode 55 and the second electrode 15.

In particular, the positions of the annular portions 541E, 542E, and 543E are changed independently of each other. Therefore, the contact pressure is prevented from increasing more than necessary for all of the first and second inner-peripheral-side electrodes 551 and 151, the first and second intermediate electrodes 552 and 152, and the first and second outer-peripheral-side electrodes 553 and 153, suppressing wear thereof.

In addition, the contact pressure is prevented from increasing more than necessary, thereby reducing contact resistance between the first electrode 55 and the second electrode 15, suppressing heat generation between the first electrode 55 and the second electrode 15.

Furthermore, in the present embodiment, the first inner-peripheral-side deformation portion 541 is provided with the openings 541C. Likewise, the openings 542C are provided in the first intermediate deformation portion 542. Furthermore, the first slit 541B is provided between the first inner-peripheral-side deformation portion 541 and the first intermediate deformation portion 542. Likewise, the first outer-peripheral-side deformation portion 543 is provided with the openings 543C. Furthermore, the second slit 542B is provided between the first intermediate deformation portion 542 and the first outer-peripheral-side deformation portion 543.

This makes it possible to achieve a structure in which the respective arm portions 541D, 542D, and 543D are readily elastically deformed with a simple structure. In addition, the first and second slits 541B and 542B make it possible to increase a creepage distance between the electrodes 551 to 553.

Furthermore, in the present embodiment, the second electrode 15 (the leaf spring portions 151B, 152B, and 153B) is elastically deformable. In other words, the elastic deformation of the second electrode 15 according to the external force changes an abutment position with the first electrode 55.

This makes it possible to effectively prevent the contact pressure between the first electrode 55 and the second electrode 15 from increasing more than necessary due to both of the elastic deformation of the deformation portions 541 to 543 and the elastic deformation of the second electrode 15.

Furthermore, according to the present embodiment, prevention of the increase in the contact pressure between the first electrode 55 and the second electrode 15 enables the increase in the manufacturing tolerances of the first support member 54 and the second support member 14, as described above.

### Other Embodiments

The embodiment for carrying out the disclosure has been described above, but it should be understood that the disclosure is not limited only to the embodiment described above.

In the embodiment described above, the ultrasound treatment tool according to the disclosure has a configuration to apply only the ultrasound energy to the target portion, but the ultrasound treatment tool is not limited thereto and may have a configuration to apply at least one of high-frequency energy and thermal energy in addition to the ultrasound energy. Here, “apply high-frequency energy to the target portion” means to apply high-frequency current to the target portion. In addition, “apply thermal energy to the target portion” means to transmit heat generated by a heater or the like to the target portion.

In the embodiment described above, in order to achieve a structure in which the arm portions 541D, 542D, and 543D are readily elastically deformed, the arm portions 541D, 542D, and 543D may be made of a material having higher flexibility than that of the annular portions 541E, 542E, and 543E. In addition, the thickness dimensions of the arm portions 541D, 542D, and 543D may be reduced relative to those of the annular portions 541E, 542E, and 543E.

In the embodiment described above, the first electrode 55 has an annular shape surrounding the center axis Ax, but the disclosure is not limited thereto, and it is preferable for at least one of the first electrode 55 and the second electrode 15 to have an annular shape.

In the embodiment described above, as in each of the deformation portions 541 to 543, a configuration in which each of the deformation portions 141 to 143 is elastically deformable according to the external force and thereby moves the position of the second electrode 15 may be adopted.

The ultrasound transducer device and the ultrasound treatment tool according to the disclosure have effects to suppress wear of the electrodes.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound transducer device removably and rotatably mounted to a casing, the ultrasound transducer device comprising:
an ultrasound transducer configured to generate ultrasound vibration to treat body tissue in a predetermined vibration direction;
a plurality of first electrodes configured to abut a plurality of second electrodes provided in the casing, each first electrode having an annular shape surrounding a rotation axis of the ultrasound transducer device, the plurality of first electrodes being annular, each of the plurality of first electrodes having different diameter dimensions; and
a plurality of first support members that includes:
a support base; and
a plurality of first deformation portions, each first deformation portion protruding from the support base along the rotation axis, the plurality of first deformation portions having annular shapes, each of the plurality of first deformation portions having different diameters; the plurality of first support members supporting the plurality of first electrodes,
wherein:
in each of the plurality of first deformation portions, a protrusion length from the support base increases as a diameter of one of the first deformation portions decreases,
a space extending over an entire periphery in a circumferential direction around the rotation axis is provided between adjacent first deformation portions, and
the first deformation portion is configured to elastically deform and move the first electrode.

2. The ultrasound transducer device according to claim 1, wherein:
the first deformation portion is provided with an opening penetrating from an outer peripheral surface to an inner peripheral surface.

3. The ultrasound transducer device according to claim 1, wherein:
the first deformation portion includes a first electrode support portion configured to abut and support the first electrode, and an arm portion configured to connect the first electrode support portion and the support base.

4. The ultrasound transducer device according to claim 3, wherein:
a radius of the arm portion is larger than a radius of the first electrode support portion.

5. The ultrasound transducer device according to claim 3, wherein:
the arm portion is made of a material having a higher flexibility than that of the first electrode support portion.

6. The ultrasound transducer device according to claim 3, wherein:
intermediate wiring electrically connected to the first electrode is provided inside the first support member.

7. An ultrasound treatment tool comprising:
an end effector configured to treat body tissue;
a casing configured to support the end effector; and
an ultrasound transducer device that is removably and rotatably mounted to the casing,
wherein the ultrasound transducer device includes:
an ultrasound transducer configured to generate ultrasound vibration to treat the body tissue in a predetermined vibration direction;
a plurality of first electrodes configured to abut a plurality of second electrodes provided in the casing, each first electrode having an annular shape surrounding a rotation axis of the ultrasound transducer device, each of the plurality of first electrodes having annular shapes with different diameters; and
a plurality of first support members that includes:
a support base; and
a plurality of first deformation portions, each first deformation portion protruding from the support base along the rotation axis so as to surround the rotation axis, each of the plurality of first deformation portions having annular shapes with different diameters, wherein:
the plurality of first support members support the plurality of first electrodes,
in each of the plurality of first deformation portions, a protrusion length from the support base increases as a diameter of one of the first deformation portions decreases,
a space extending over an entire periphery in a circumferential direction around the rotation axis is provided between adjacent first deformation portions, and
the first deformation portion is configured to elastically deforms to move the first electrode.

8. The ultrasound treatment tool according to claim 7, wherein:
the first deformation portion is provided with an opening penetrating from an outer peripheral surface to an inner peripheral surface.

9. The ultrasound treatment tool according to claim 7, the first deformation portion including:
a first electrode support portion configured to abut and support the first electrode; and
an arm portion configured to connect the first electrode support portion and the support base.

10. The ultrasound treatment tool according to claim 9, wherein:
a radius of the arm portion is larger than a radius of the first electrode support portion.

11. The ultrasound treatment tool according to claim 9, wherein:
the arm portion is made of a material that is more flexible than a material of the first electrode support portion.

12. The ultrasound treatment tool according to claim 9, wherein:
intermediate wiring electrically connected to the first electrode is provided inside the first support member.
